(19) United States
(12) Patent Application Publication
(10) Pub. No.: US 2025/0334656 A1
(43) Pub. Date: Oct. 30, 2025

(54) APPARATUS AND METHOD FOR MAGNETIC RESONANCE IMAGING

(71) Applicant: The Penn State Research Foundation, University Park, PA (US)

(72) Inventors: Parisa Lotfi Poshtgol, Hershey, PA (US); Michael Lanagan, University Park, PA (US); Qing Yang, Hershey, PA (US); Federico Krauch, University Park, PA (US); Thomas Neuberger, University Park, PA (US)

(21) Appl. No.: 19/189,712

(22) Filed: Apr. 25, 2025

Related U.S. Application Data
(60) Provisional application No. 63/638,612, filed on Apr. 25, 2024.
(60) Provisional application No. 63/638,679, filed on Apr. 25, 2024.

(51) Int. Cl. G01R 33/341; A61B 5/055

(52) U.S. Cl. CPC G01R 33/341; A61B 5/055

(57) ABSTRACT

Embodiments relate to apparatuses, methods, and systems configured to increase at least one of radiofrequency transmission efficiency and/or radiofrequency receiving sensitivity. In particular, embodiments relate to a magnetic imaging apparatus having at least one spiral coil that provides increased radiofrequency transmission efficiency and/or radiofrequency receiving sensitivity.

### CROSS-REFERENCE TO RELATED APPLICATIONS

This patent application is related to and claims the benefit of priority of U.S. Provisional Application 63/638,612, filed on Apr. 25, 2024, and U.S. Provisional Application 63/638,679, filed on Apr. 25, 2024, the entire contents of which are incorporated by reference.

### STATEMENT REGARDING FEDERALLY SPONSORED RESEARCH OR DEVELOPMENT

This invention was made with government support under Grant Nos. EB026978 and EB027061 awarded by the National Institutes of Health. The Government has certain rights in the invention.

### FIELD

Embodiments relate to apparatuses, methods, and systems configured to provide magnetic resonance imaging (MRI). In particular, embodiments relate to an MRI apparatus having a spiral coil with multiple loops and configured to provide increased radiofrequency (RF) energy transmission efficiency, increased RF receiving sensitivity, and reduced electrical noise in MRI applications.

### BACKGROUND

Magnetic resonance imaging (MRI) is a well-known technique that can be used to generate highly detailed images of matter having atoms with MRI-compatible nuclei, such as hydrogen, sodium, oxygen, etc. For example, MRI can be used to generate images identifying anatomical structure, physiological processes, tissue viability, etc., and has a wide range of imaging applications. MRI utilizes various types of electrically conductive magnets and coils to generate static and oscillating magnetic fields. Matter is subjected to the magnetic fields such that a precession (e.g., a movement) of the magnetic moment of the nuclei of the MRI-compatible atoms within the matter aligns in a specific orientation relative to one or more of the magnetic fields. Radiofrequency (RF) energy signals are emitted from the atoms during a re-aligning precession of the atoms, which can be processed into highly detailed images of the matter.

### SUMMARY

One electrically conductive coil used in MRI applications is a radiofrequency (RF) coil. In an MRI application, one or more RF coils are used to transmit RF energy, in the form of a pulsed oscillating magnetic field, to the atomic nuclei such that the precession alignment of the nuclei is altered during the pulses. As the nuclei return to their non-altered precession alignment, the nuclei emit RF energy signals that are received by the same, or alternative, RF coil(s). Information about the nuclei's movement during realignment is extracted from the RF signals received by the RF coil(s) and mathematically processed, e.g., with Fourier Transform equations, to construct highly detailed images of the matter based on the received RF signals.

The RF energy transmission efficiency and RF energy receiving sensitivity of the RF coil(s) in an MRI application are crucial to the ability to generate highly detailed and accurate images. Additionally, the RF signals emitted from the nuclei can be weak signals that can be significantly affected by electrical noise, which can further impede the ability to generate highly detailed and accurate images and can lead to longer MRI scan times for patients. As such, increasing RF coil energy transmission efficiency and receiving sensitivity, and reducing electrical noise in RF signals received by the RF coil(s) is of particular interest in MRI applications.

We have developed a multi-loop spiral coil for increasing RF energy transmission efficiency, increasing RF energy receiving sensitivity, and reducing electrical noise. The coil consists of at least a first loop and a second loop that are positioned concentrically with respect to a central axis. The coil may function by reducing an electric field in a sample (e.g., matter to be imaged) such that electrical noise in the sample is reduced, and RF energy transmission efficiency and receiving sensitivity are increased.

Our multi-loop spiral coil demonstrates an increase in RF energy transmission efficiency and receiving sensitivity and a reduction of electrical noise, as compared to conventional RF coils in MRI applications, such as a single-loop coil. Therefore, our multi-loop spiral coil highlights the opportunity to improve MRI applications through improved image quality, reduced scan times, and increased patient throughput.

In an exemplary embodiment, a magnetic resonance imaging apparatus comprises at least one spiral coil comprising a first loop and a second loop, wherein the first loop and the second loop are positioned concentrically with respect to a central axis and spaced apart to define a gap intermediate the first loop and the second loop; wherein the at least one spiral coil is configured to reduce its conservative electric field induced in a sample to be imaged such that at least one of a radiofrequency transmission efficiency or a radiofrequency receiving sensitivity of the apparatus is increased.

In some embodiments, reducing the conservative electric field induced in the sample comprises confining the conservative electric field between the first loop and the second loop such that electric field generated within the sample is dominantly non-conservative.

In some embodiments, the conservative electric field is confined within the gap defined intermediate the first loop and the second loop.

In some embodiments, the at least one spiral coil is further configured to reduce a noise level in the subject.

In some embodiments, the apparatus comprises a plurality of spiral coils.

In some embodiments, the apparatus comprises at least one high dielectric constant material disk disposed within the at least one spiral coil, wherein the at least one high dielectric constant material disk is configured to increase at least one of the radiofrequency transmission efficiency or the radiofrequency receiving sensitivity of the magnetic resonance imaging apparatus.

In some embodiments, the high dielectric constant material disk comprises a disk resonator with a disk resonance frequency equal to a coil resonance frequency of the at least one spiral coil.

In some embodiments, the high dielectric constant material disk comprises at least one ultrahigh dielectric material.

In an exemplary embodiment, a method for increasing at least one of a radiofrequency receiving sensitivity or radiofrequency transmission efficiency of a magnetic resonance imaging apparatus comprises providing the magnetic resonance imaging apparatus comprising at least one spiral coil comprising a first loop and a second loop, wherein the first loop and the second loop are positioned concentrically with respect to a central axis and spaced apart to define a gap intermediate the first loop and the second loop, wherein the at least one spiral coil is configured to reduce its conservative electric field induced in a sample such that at least one of the radiofrequency transmission efficiency or the radiofrequency receiving sensitivity of the magnetic resonance imaging apparatus is increased; transmitting, via the spiral coil, radiofrequency energy to the sample; and/or receiving, via the spiral coil, radiofrequency energy from the sample.

In some embodiments, reducing the conservative electric field induced in the sample comprises confining the conservative electric field induced by the at least one spiral coil between the first loop and the second loop such that a dominantly non-conservative electric field is generated within the sample.

In some embodiments, the conservative electric field is confined within the gap defined intermediate the first loop and the second loop.

In some embodiments, the magnetic resonance imaging apparatus further comprises a plurality of spiral coils.

In some embodiments, the magnetic imaging apparatus further comprises at least one high dielectric constant material disk disposed within the at least one spiral coil, wherein the at least one high dielectric constant material disk is configured to increase at least one of the radiofrequency transmission efficiency or the radiofrequency receiving sensitivity of the magnetic resonance imaging apparatus.

In some embodiments, the high dielectric constant material disk comprises a disk resonator with a disk resonance frequency equal to a coil resonance frequency of the at least one spiral coil.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is an illustration of a top view of an exemplary spiral coil comprising a first loop and a second loop.

FIG. 2 is an illustration of a perspective view of an exemplary spiral coil.

FIG. 3 is an illustration of a top view of an exemplary spiral coil with a high (or ultrahigh) dielectric constant material disposed therein.

FIG. 4 is a schematic block diagram of an exemplary embodiment of a magnetic resonance imaging apparatus.

FIG. 5 is a schematic block diagram of an exemplary embodiment of a magnetic resonance imaging apparatus.

FIG. 6 is a schematic block diagram of an exemplary embodiment of a magnetic resonance imaging apparatus.

FIG. 7 is a schematic block diagram of an exemplary embodiment of a magnetic resonance imaging apparatus.

FIG. 8 is a flow chart illustrating an exemplary method for increasing at least one of a radiofrequency receiving sensitivity or radiofrequency transmission efficiency of a magnetic resonance imaging apparatus.

FIG. 9A is an illustration of a simulation setup including a coil with one loop.

FIG. 9B is an illustration of a simulation setup including a coil with one loop and a cylindrical water phantom and a uHDCM disk disposed therein.

FIG. 9C is an illustration of a simulation setup including a coil with two loops and a cylindrical water phantom and a uHDCM disk disposed therein.

FIG. 10A shows an electric field map generated from the coil with one loop shown in FIG. 9.

FIG. 10B shows an electric field map generated from the coil with one loop and a cylindrical water phantom and a uHDCM disk disposed therein shown in FIG. 9.

FIG. 11A shows an electric field map generated from a coil with two loops.

FIG. 11B shows an electric field map generated from the coil with two loops and a cylindrical water phantom and a uHDCM disk disposed therein shown in FIG. 9.

FIG. 12 is a line pot showing a reduction of conservative electric fields among the tested coils.

FIG. 13 is a graph showing Rx-sensitivity for tested coils.

FIG. 14 is an illustration of a perspective view of an exemplary spiral coil.

### DETAILED DESCRIPTION

The following description is of exemplary embodiments that are presently contemplated for carrying out the present invention. This description is not to be taken in a limiting sense but is made merely for the purpose of describing the general principles and features of the present invention. The scope of the present invention is not limited by this description.

Embodiments generally relate to apparatuses, methods, and systems configured to provide increased radiofrequency (RF) transmission efficiency, increased RF receiving sensitivity, and reduced electrical noise in magnetic resonance imagining (MRI) applications. In particular, a magnetic resonance imaging apparatus can be configured to provide such increased radiofrequency (RF) transmission efficiency, increased RF receiving sensitivity, and reduced electrical noise and can include at least one spiral coil having a first loop and a second loop positioned concentrically with respect to a central axis and configured to reduce an electric field in a sample.

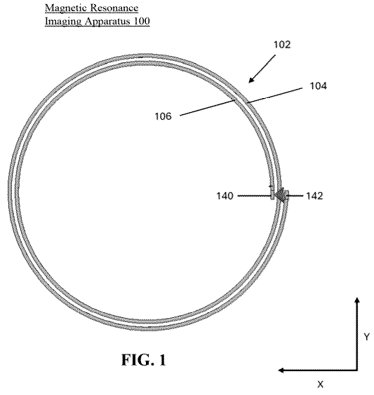

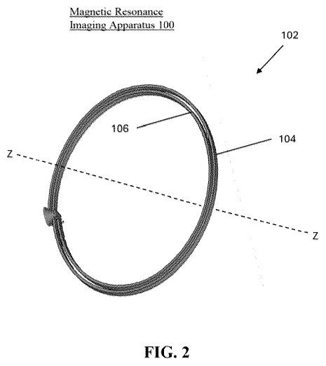

Referring to FIGS. 1-4, a magnetic resonance imagining (MRI) apparatus 100 includes a spiral coil 102. In some embodiments, the MRI apparatus 100 can include a plurality of spiral coils 102. In some embodiments, the plurality of spiral coils 102 can be arranged as an array of spiral coils 102. The spiral coil(s) 102 can be radiofrequency (RF) coil(s). The spiral coil(s) 102 can include a first loop 104 and a second loop 106. The first loop 104 can be spaced apart from and parallel to the second loop 106. In some embodiments, the spiral coil(s) 102 can include more than two loops. For example, in one embodiment, the spiral coil(s) 102 can include a first, second, and third loop. In another embodiment, the spiral coil(s) can include a first, second, third, and fourth loop, etc.

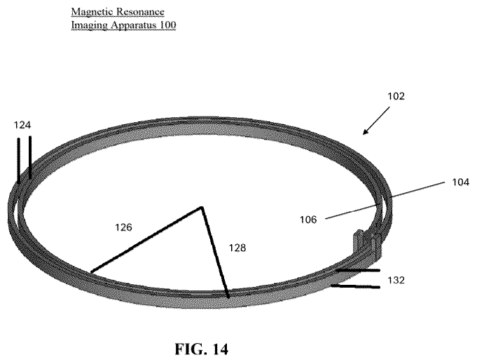

The spiral coil(s) 102 can include a first end 140 and a second end 142. The spiral coil(s) 102 can be continuously formed from the first end 140 to the second end 142. As shown in FIG. 14, the first loop 104 and the second loop 106 can be spaced apart from each other such that a gap 124 is defined intermediate the first loop 104 and the second loop 106. The first loop 104 can have a first radius 128 and the second loop 106 can have a second radius 126. The spiral coil(s) 102 can have a height 132.

The spiral coil(s) 102 can include any conductive material suitable for transmitting and/or receiving RF energy, such as copper, silver, aluminum, superconductive materials (e.g., niobium-titanium), Litz wire, etc.

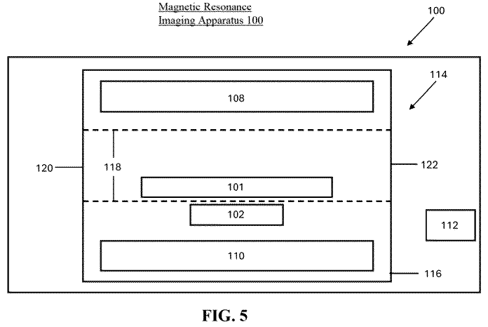

The MRI apparatus 100 can be configured to transmit and/or receive electrical energy, such as RF energy, to and/or from a sample 101 (FIGS. 5-7, 9C, and 11A-B). In some embodiments, the MRI apparatus 100 can include one or more spiral coils 102 configured to transmit RF energy to the sample 101. In some embodiments, the MRI apparatus 100 can include one or more spiral coils 102 configured to receive RF energy from the sample 101, such as RF energy signals emitted by the sample 101 during an MRI analysis of the sample 101. In some embodiments, the MRI apparatus 100 can include one or more spiral coils 102 configured to both transmit and receive RF energy to and from the sample 101.

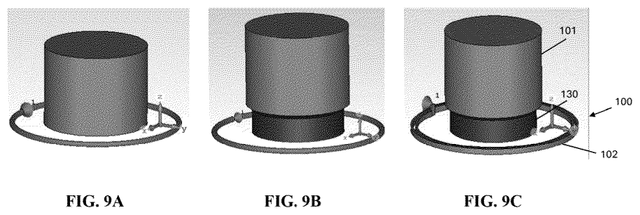

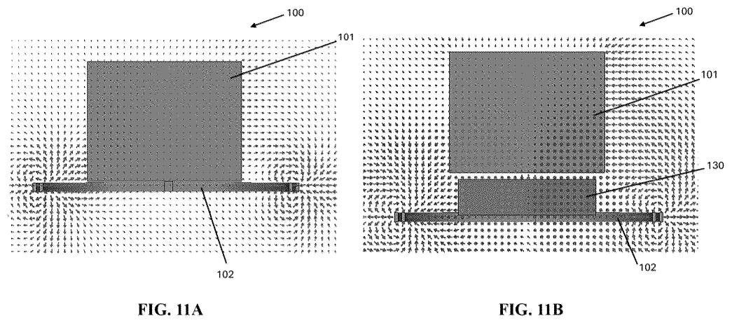

The sample 101 can include any matter suitable for MRI analysis, such as human tissue, animal tissue, plant tissue, liquids, gels, plastics, etc. In some embodiments, the sample 101 can include a phantom, such as a water phantom (FIGS. 9C and 11A-B).

The MRI apparatus 100, and/or elements thereof, can be in electrical connection/communication with at least one power supply such that electrical energy can be transmitted from the power supply to the MRI apparatus 100 and/or elements thereof, as described below. In some embodiments, the power supply can be included in the MRI apparatus 100. In some embodiments, the power supply can be external to the MRI apparatus 100. In some embodiments, a plurality of power supplies can be in electrical connection/communication with the MRI apparatus 100 and/or elements thereof.

For example, the MRI apparatus 100 can receive an alternating current from the at least one power supply, and the alternating current can flow through the spiral coil(s) 102 such that the MRI apparatus 100, via the spiral coil(s) 102, can generate an oscillating magnetic field, which can be referred to as magnetic field B1. The alternating current, and thus, the oscillating magnetic field B1 generated by the flow of the alternating current, can be in the RF range. The MRI apparatus 100 can be configured to generate the RF oscillating magnetic field B1 such that the RF energy of B1 is transmitted to the sample 101. For example, the RF oscillating magnetic field can be generated within and/or adjacent the MRI apparatus 100, such that the sample 101 is exposed to and/or penetrated by oscillating magnetic field B1 such that RF energy from B1 can couple with the magnetic moment of atomic nuclei within the sample 101.

In an exemplary MRI analysis of the sample 101, the sample 101 is exposed to a plurality of magnetic fields, each of the magnetic fields influencing the alignment, or precession, of atomic nuclei within the sample 101. For example, a static magnetic field, which can be referred to as magnetic field B0, can be generated such that a precession of the magnetic moment of each of the atomic nuclei of the sample 101 within the magnetic field B0 aligns within with the static magnetic field B0. For example, the precession of the magnetic moment of the atomic nuclei can align in a parallel orientation relative to the magnetic field B0, which can be referred to a low-energy nuclear spin state.

The magnetic moment of the atomic nuclei precesses at a specific frequency known as the Larmor frequency. The Larmor frequency for a given atomic nuclei depends on the type of atom, e.g., hydrogen atom, sodium atom, oxygen atom, etc. The coupling of RF energy from B1 to the atomic nuclei magnetic moments can excite the nuclei, which can induce the precession into a high-energy nuclear spin state and tip the net magnetization vector of the nuclei into anti-parallel alignment with B0. The MRI apparatus 100 can be configured to generate B1 at or near the Larmor frequency to optimize RF transmission efficiency and/or receive sensitivity.

The oscillating magnetic field B1 can be generated in pulses, and thus, RF energy can be transmitted from the MRI apparatus 100 to the sample 101 in pulses. In between pulses of B1 generated by the MRI apparatus 100, the atomic nuclei can emit RF energy signals as they relax from the high energy nuclear spin state alignment with magnetic field B0 to the low-energy nuclear spin state alignment with magnetic field B0. The MRI apparatus 100, for example, via the spiral coil(s) 102, can receive the RF energy signals emitted by the atomic nuclei during the relaxation/realignment with B0. The RF signals received by the MRI apparatus 100 can be processed into detailed images of the sample 101, as further described below.

The oscillating magnetic field B1 can be said to have two components: B1+, which is a component of B1 that is associated with the transmission of RF energy from the spiral coil(s) 102 to atomic nuclei of the sample 101, and B1−, which is a component of B1 associated with the reception of RF energy signals emitted from the atomic nuclei and received by the spiral coil(s) 102. The transmission efficiency of B1+, that is, the efficiency with which RF energy is transmitted from the spiral coil(s) 102 to the atomic nuclei of the sample 101, and the receive sensitivity of B1−, that is, the ability of the spiral coil(s) 102 to receive the RF signals emitted by the atomic nuclei of the sample 101, can both play a crucial role in generating high quality and accurate MRI images of the sample 101, as well as reducing overall scan times in MRI applications.

In MRI applications, electric field(s) within a sample (e.g., sample 101), such as a conservative electric field generated by the spiral coil(s) 102, can result in imaging noise and/or can negatively impact the transmission efficiency (B1+) and/or receive sensitivity (B1−) of RF coil(s), such as the spiral coil(s) 102 of the MRI apparatus 100. For example, the presence of conservative electric fields within the sample 101 can decrease the signal-to-noise ratio (SNR), which can decrease the receive sensitivity of RF coil(s). Reducing the conservative electric field, such as by confining the conservative electric field to the spiral coil(s) 102 can reduce dielectric losses and/or enhance inductive coupling between the spiral coil(s) 102 and the sample 101, which can increase the SNR. In other words, confining the conservative electric field to the spiral coil(s) 102 can increase the SNR, and thus, increase the receive sensitivity of the RF coil(s) 102.

Further, confining the conservative electric field to the spiral coil(s) 102 can increase the RF transmission efficiency of the spiral coil(s) 102. For example, confining the conservative electric field within the spiral coil(s) 102 (e.g., confining the conservative electric field between the first loop and the second loop) can enhance inductive coupling between the spiral coil(s) 102 and the sample 101. Enhancing the inductive coupling between the spiral coil(s) 102 and the sample 101 can reduce energy losses in the sample 101, by reducing or eliminating displacement currents in the sample 101, such that more of the RF energy (e.g., RF electrical power) supplied to the spiral coil(s) 102 is directed to generating a more homogenous, focused, and/or stronger B1+ oscillating magnetic field. The resulting enhancement(s) (e.g., homogeneity, focus, strength, etc.) can provide more uniform and/or deeper penetration of the oscillating magnetic field B1+ into the sample 101.

Reducing the conservative electric field in the sample 101, such as by confining the conservative electric field to the spiral coil(s) 102, can generate a dominantly non-conservative electric field, generated through the oscillating magnetic field B1+, in the sample 101. Generating a dominantly non-conservative field within the sample 101 can further increase RF transmission efficiency of the spiral coil(s) 102. For example, generating a dominantly non-conservative field within the sample 101 can provide stronger and/or more efficient excitation and/or improved flip angle control of the nuclei of the sample 101. In other words, reducing or minimizing the conservative electric field in the sample 101 to reduce electric losses and create a dominantly non-conservative electric field in the sample 101 can increase and/or enhance the SNR, transmission efficiency, and/or receive sensitivity of spiral coil(s) 102 of the MRI apparatus 100.

In an exemplary embodiment, the spiral coil(s) 102 can be configured to reduce a conservative electric field (e.g., a conservative electric field generated by the spiral coil(s) 102) in the sample 101 such that the RF transmission efficiency, and/or RF receive sensitivity of the MRI apparatus 100 increases. For example, the first loop 104 can be spaced apart from and parallel to the second loop 106 to define a gap 124, such that a conservative electric field can be confined between the first loop 104 and the second loop 106 within the gap 124 to reduce the electric field within a sample 101 adjacent and/or within the MRI apparatus 100.

The size of the gap 124 (e.g., width of the space between the first loop 104 and the second loop 106) can depend on operational and/or geometric parameters of the spiral coil(s) 102 and/or the MRI apparatus 100. The size of the gap 124 can depend on a desired operating frequency (e.g., Larmor frequency), the sample 101 to be imaged (e.g., the type of MRI application), a dielectric material of the MRI apparatus 100 (described below), the overall size (e.g., diameter) of the spiral coil(s) 102, etc. For example, larger diameter spiral coil(s) 102 can require a different gap size than smaller diameter spiral coil(s) 102 to maintain desired resonance and/or impedance characteristics of the MRI apparatus 100.

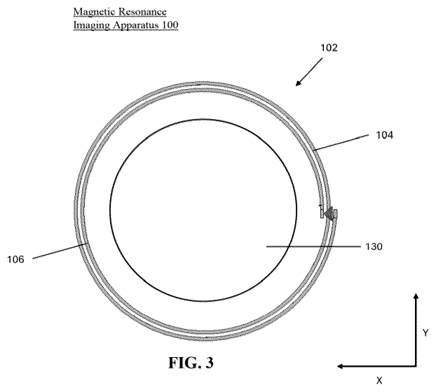

Referring now to FIGS. 3, 6, 9C, and 11B, in some embodiments, the MRI apparatus 100 can include one or more high, or ultrahigh dielectric constant material disks 130. The one or more high or ultrahigh dielectric constant disks can be high or ultrahigh dielectric material disk resonators 130. For example, the one or more uHDCM disks 130 can be uHDCM disk resonators 130 configured to have a predetermined disk resonance frequency. In other words, the uHDCM disk resonators 130 can be configured (e.g., shaped, sized, materially constructed, etc.) to resonate at a predetermined frequency, such as an operational frequency of the spiral coil(s) 102 (e.g., Larmor frequency). It should be understood that element 130, hereinafter referred to as uHDCM disk resonator 130, can refer to a high dielectric constant (e.g., dielectric constant, or relative permittivity, in a range from 1,000 to 5,000) material (HDCM) disk resonator or an ultrahigh dielectric constant (e.g., dielectric constant, or relative permittivity, in a range from 5,000 to 10,000) material (uHDCM) disk resonator.

In some embodiments, the uHDCM disk resonator(s) 130 can include a tunable ultrahigh dielectric constant ceramic (tuHDC) disk resonator 130. The uHDCM disk resonator(s) 130 can be disposed within and/or adjacent the spiral coil(s) 102. For example, the uHDCM disk resonator(s) 130 can be centrally disposed within and/or above or below the spiral coil(s) 102. The uHDCM disk resonator(s) 130 can be disposed within and/or adjacent the spiral coil(s) 102 such that the uHDCM and the spiral coil(s) 102 couple to each other, for example, in a capacitive (also referred to as anti-parallel) coupling mode or an inductive (also referred to as parallel) coupling mode. The uHDCM disk resonator(s) 130 can be configured to enhance the magnetic field B1 generated by the MRI apparatus 100, to reduce hotspots in the sample 101, and/or to manage the specific absorption rate (SAR) of the sample 101.

For example, the uHDCM disk resonator(s) 130 can be configured to induce and/or increase induced displacement current(s) within the uHDCM disk resonator(s) 130, the displacement current(s) being induced by a non-conservative electric field induced by the spiral coil(s) 102, e.g., a non-conservative electric field generated by the oscillating magnetic field B1 generated by the spiral coil(s) 102. Induced displacement currents within the uHDCM disk resonator(s) 130 can generate secondary oscillating magnetic B1+ fields that can constructively interfere with the B1+ field generated by the spiral coil(s) 102, such that the overall B1+ field that penetrates the sample 101 can have increased strength and/or focus within the sample 101. In other words, the uHDCM disk resonator(s) 130 can focus, shape, and increase the strength of the B1+ field within the sample 101 such that the transmission efficiency and/or receive sensitivity of the spiral coil(s) 102, and thus of the MRI apparatus 100, is increased.

The uHDCM disk resonator(s) 130 can include any suitable high or ultrahigh dielectric material(s). The dielectric materials of the uHDCM disk resonator(s) 130 can depend on coil geometry of the spiral coil(s) 102, a desired operating frequency and/or resonant frequency, physical properties (e.g., size, spatial constraints, shape, etc.) of the MRI apparatus 100 and/or elements thereof, or any other operational parameters. In some embodiments, the dielectric materials of the uHDCM disk resonator(s) 130 can depend on a desired high or ultrahigh relative permittivity, or dielectric constant. For example, a high relative permittivity, or dielectric constant, can be a relative permittivity in a range from 1,000 to 5,000. An ultrahigh relative permittivity, or dielectric constant, can be a relative permittivity in a range from 5,000 to 10,000, etc. In some embodiments, an ultrahigh relative permittivity, or dielectric constant, can be greater than 10,000. Suitable dielectric materials can include any suitable dielectric materials having a high or ultrahigh relative permittivity, or dielectric constant, such as specialized ceramics, barium titanate, barium strontium titanate, such as Ba(x)Sr(1-x)TiO3, with 0.3<x<0.7, etc.

In some embodiments, the uHDCM disk resonator(s) 130 can include a plurality of uHDCM resonator disks or layers. For example, the uHDCM disk resonator(s) can include a first uHDCM resonator disk and a uHDCM resonator disk. Each of the uHDCM resonator disks can be a different dielectric material, the same dielectric material, or combinations thereof (e.g., a first resonator disk and a second resonator disk can be the same material and a third resonator disk can be a different material, etc.). In some embodiments, each of the uHDCM resonator disks can have a different relative permittivity value, the same relative permittivity value, or combinations thereof.

In some embodiments, the uHDCM disk resonator(s) 130 can include a plurality of resonator disks or layers configured to reduce a frequency temperature coefficient such that a resonant frequency variation of the uHDCM disk resonator(s) 130 is reduced and/or a desired resonant frequency (e.g., Larmor frequency, operational frequency of the spiral coil(s) 102, etc.) of the uHDCM disk resonator(s) 130 is stabilized across a range of temperatures. For example, the uHDCM disk resonator(s) 130 can include a first resonator disk composed of a dielectric material having a peak relative permittivity at a first temperature, and a second resonator disk composed of a dielectric material having a peak relative permittivity at a second temperature that is different from the first temperature. In some embodiments, the first resonator disk can have a negative frequency temperature coefficient and the second resonator disk can have a positive frequency temperature coefficient, or any combination thereof.

Configuring the uHDCM disk resonator(s) 130 to include a first resonator disk having a peak relative permittivity at a first temperature and a second resonator disk having a peak relative permittivity at a second, different, temperature can average out, or stabilize, the relative permittivity of the uHDCM disk resonator(s) 130 such that the resonant frequency of the uHDCM disk resonator(s) 130 is stabilized across a range of temperatures. In other words, in such embodiments, the frequency temperature coefficient can be reduced such that the resonant frequency variation of the uHDCM disk resonator(s) 130 is reduced. In some embodiments, the plurality of resonator disks or layers can be configured to tune the uHDCM resonator(s) 130 to a desired resonant frequency.

The spiral coil(s) 102 and the uHDCM disk resonator(s) 130 each have a resonant frequency (e.g., a coil resonant frequency and a disk resonator resonant frequency, respectively) that can be determined by one or more physical and/or operational parameters. For example, the size, shape, material, geometric configuration, etc., of the spiral coil(s) 102 and/or uHDCM disk resonator(s) 130 can be configured based on a desired resonant frequency. In some embodiments, the spiral coil(s) 102 can be physically configured (e.g., size of the gap 124, radius 126/128, height 132, conductive material, etc.) based on a desired resonant frequency, such as an operational Larmor frequency. The uHDCM disk resonator(s) 130 can be physically configured (e.g., size, shape, dielectric material, etc.) based on a desired resonant frequency at or near the resonant frequency of the spiral coil(s) 102, such that spiral coil(s) 102 and the uHDCM disk resonator(s) 130 operate at or near the same resonant frequency.

In some embodiments, the MRI apparatus 100 and/or elements thereof (e.g., the spiral coil(s) 102) can be shaped, sized, etc., based on an application of the MRI apparatus 100. For example, the MRI apparatus 100 and/or elements thereof can be configured, e.g., shaped, sized, arranged, etc., based on the type of MRI being conducted, e.g., a hydrogen MRI, oxygen MRI, sodium MRI, etc. The MRI apparatus 100 and/or elements thereof can be configured based on the area or portion(s) of a sample 101 to be imaged, such as a torso, limb, extremity, head, etc.

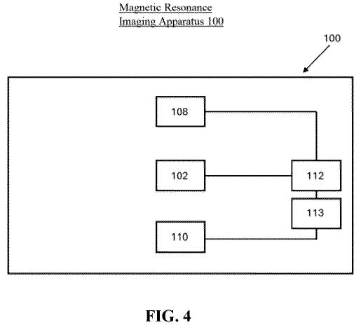

Referring now to FIG. 4A and FIG. 4B, in some embodiments, the MRI apparatus 100 can include additional elements utilized in magnetic resonance imaging applications. For example, in some embodiments, the MRI apparatus 100 can include at least one magnet 108 configured to generate a first magnetic field, such as static magnetic field B0. The first magnetic field can be generated within and/or adjacent the MRI apparatus 100, such that the sample 101 is exposed to and/or penetrated by the magnetic field B0. The magnet(s) 108 can be any suitable magnet(s) for generating the first magnetic field, such as superconductive magnet(s), resistive magnet(s), permanent magnet(s), etc. The magnet(s) 108 can be configured to generate a B0 magnetic field having a specific magnetic field strength measured in units of Tesla (T). The magnetic field strength of B0 can depend on operational parameters such as the type of MRI being conducted, the desired image quality, the technical specifications of one or more elements of the MRI apparatus 100, etc.

For example, in some embodiments, the B0 magnetic field strength can be in a range from 1T to 3T, in a range from 3T to 7T, and/or in a range from 7T to 12T. In some embodiments, the B0 magnetic field strength can be greater than 12T. The MRI apparatus 100, and/or elements thereof (e.g., the spiral coil(s) 102, the magnet(s) 108, etc.) can be configured (e.g., sized, shaped, arranged, etc.) based on the desired magnetic field strength of B0.

In some embodiments, the MRI apparatus 100 can include at least one gradient coil 110. The gradient coil(s) 110 can include any suitable electromagnetic coil(s) configured to generate at least one secondary magnetic field, such as a gradient magnetic field. In some embodiments, the gradient coil(s) 110 can be configured to generate a plurality of gradient magnetic fields. The gradient magnetic field(s) can be generated within and/or adjacent the MRI apparatus 100, such that the sample 101 is exposed to and/or penetrated by the magnetic field B0. The gradient magnetic field(s) can cause the Larmor frequency of atomic nuclei precession to vary based on the position of the nuclei, which can provide spatial encoding of RF energy signals emitted from the atomic nuclei of the sample 101. In other words, the gradient magnetic field(s) can provide a precise selection of a portion, or slice, of the sample 101 to excite via the oscillating magnetic field B1, and thus, a desired slice of the sample 101 can be imaged. Strength and/or speed of the gradient magnetic field(s) can influence the resolution and/or field of view of the imaging.

In some embodiments, the MRI apparatus 100 can include at least one computing device 112. In some embodiments, the computing device(s) 112 can be external to the MRI apparatus 100. The computing device(s) 112 can have sufficient processing power and input and output devices for generating magnetic resonance images. For example, the computing device(s) 112 can be in electrical communication and/or connection with the MRI apparatus 100 and/or elements thereof (e.g., spiral coil(s) 102, magnet(s) 108, gradient coil(s) 110, power supply, electrical circuits/components, etc.) such that data can be transmitted to and/or from the computing device(s) 112 and the MRI apparatus 100 and/or elements thereof.

The MRI apparatus 100 may be configured to continuously transmit data to the computing device(s) 112, or the MRI apparatus 100 may be configured to periodically transmit data to the computing device(s) 112 (e.g., non-continuously at pre-determined intervals, such as every five seconds, every 10 seconds, etc.). In some embodiments, the MRI apparatus 100 may provide real-time data to the computing device(s) 112. The transmission of the data can be provided via a communication connection the MRI apparatus 100 directly has with the computing device(s) 112.

It should be appreciated that the computing device(s) 112 may be a computer device that may have at least one processor (Proc) connected to at least one non-transitory computer readable medium (e.g. memory, a hard drive, etc.) (Mem) and at least one transceiver unit (e.g. a network transceiver, etc.) (Trcvr). The computing device(s) 112 may be a desktop computer, laptop computer, smart phone, tablet, testing instrument (e.g., oscilloscope), or any other suitable device.

The computing device(s) 112 may be configured to store the sensor data transmitted by the MRI apparatus 100, such as within its non-transitory computer readable medium.

In some embodiments, sensor data may alternatively or subsequently be transmitted to a central computer device 113. The central computer device 113 may be operatively connected to the computing device(s) 112 (e.g., via a network connection and application programming interface (API) the central computer device 113 may have with the computing device(s) 112 running the application, etc.) and/or the MRI apparatus 100. The transmission of the data can be provided via a communication connection the MRI apparatus 100 directly has with the central computer device 113, or via a communication connection the MRI apparatus 100 directly has with a computing device(s) 112 such that data is transmitted to the central computer device 113 via the computing device(s) 112. The MRI apparatus 100 and/or computing device(s) 112 may be configured to continuously transmit data to the central computer device 113, or the sensor 100 and/or input/output device 200 may be configured to periodically transmit data to the central computer device 113 (e.g., non-continuously at pre-determined intervals, such as every five seconds, every 10 seconds, etc.). In some embodiments, the MRI apparatus 100 and/or computing device(s) 112 may provide real-time data to the central computer device 113.

It should be appreciated that the central computer device 113 can be configured as a computer device that may have at least one processor (Proc) connected to at least one non-transitory computer readable medium (e.g. memory, a hard drive, etc.) (Mem) and at least one transceiver unit (e.g. a network transceiver, etc.) (Trcvr). The central computer device 113 can be configured as a database server, a cloud-based server, an operator workstation, a desktop computer, a laptop computer, or any other suitable computer device.

The central computer device 113 may be configured to store the sensor data transmitted by the MRI apparatus 100 and/or computing device(s) 112, such as within its non-transitory computer readable medium.

The computing device(s) 112 and/or the central computer device 113 may be configured to analyze the data. In some embodiments, analysis of the sensor data may include generate MRI images based on data received from the MRI apparatus 100.

In some embodiments, the computing device(s) 112 and/or the central computer device 113 may comprise a graphical user interface (GUI) or some other communication module for providing information to the user regarding the data and/or analysis of the data and/or images based on the received data. For example, the sensor 100 may transmit sensor data to the computing device(s) 112 and/or the central computer device 113 for data analysis, and the data itself and/or analysis performed by the computing device(s) 112 and/or the central computer device 113 can subsequently be displayed to the user via a GUI. In some embodiments, the data and/or the performed analysis may be displayed in the form of a graph, chart, text, or other display via the GUI.

In some embodiments, the computing device(s) 112 and/or the central computer device 113 may use artificial intelligence or machine learning algorithms stored on the computing device(s) 112 and/or the central computer device 113. In particular, the computing device(s) 112 and/or central computer device 113 may run a program that uses the collected data along with a module trained via a machine learning process that received the collected data and processes that data to determine an output and/or generate images.

In some embodiments, the computing device(s) 112 and/or the central computer device 113 may be configured to run that code to control operation of the MRI apparatus 100.

In some embodiments, the computing device(s) 112 and/or the central computer device 113 may receive data from the MRI apparatus 100 and provide operational instructions to the MRI apparatus 100 based on the data.

Referring now to FIGS. 5-7, in some embodiments, the MRI apparatus 100 can be part of a MRI apparatus structure 114. The structure 114 can include a chamber 116. The chamber 116 can define a cavity 118 extending from a first end 120 of the chamber 116 to a second end 122 of the chamber 116. The spiral coil(s) 102, magnet(s) 108, and/or gradient coil(s) 110 can be disposed within the chamber 116 such that magnetic field(s) generated by the spiral coil(s) 102, magnet(s) 108, and/or gradient coil(s) 110 can be generated within the cavity 118. For example, in some embodiments, the chamber 116 can be an annular chamber and the spiral coil(s) 102, magnet(s) 108, and/or gradient coil(s) 110 can be disposed within the chamber 116 concentrically about the cavity 118. In such embodiments, the cavity 118 can be an annular cavity 118. The sample 101 can be disposed within the cavity 118 such that the sample 101 can be exposed to and/or penetrated by the magnetic field(s) generated by the spiral coil(s) 102, magnet(s) 108, and/or gradient coil(s) 110.

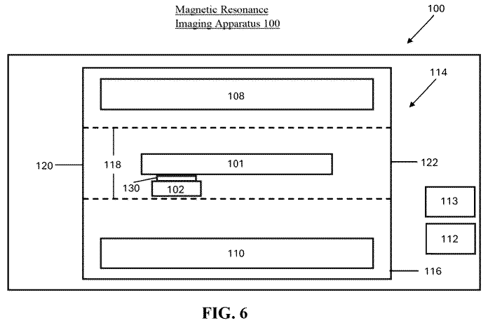

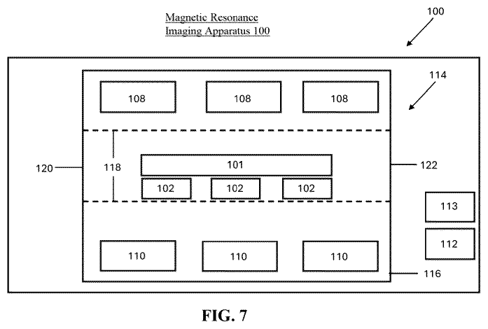

Referring now to FIGS. 6-7, in some embodiments, the spiral coil(s) 102 can be surface coil(s) disposed adjacent, e.g., above, below, next to, etc., the sample 101. In other words, the spiral coil(s) 102 can be surface coil(s) placed beneath, on top of, or beside the sample 101 such that a specific portion or area of the sample 101 can be precisely imaged. In such embodiments, the uHDCM disk resonator(s) 130 can be disposed within and/or adjacent the spiral coil(s) 102 such that the uHDCM disk resonator(s) 130 can be disposed adjacent the sample 101.

In some embodiments, the MRI apparatus 100 can include a plurality of each of the spiral coils 102, uHDCM disk resonator(s) 130, magnets 108, and/or gradient coils 110. In some embodiments, one or more spiral coil(s) 102 can be disposed within the cavity 118, and one or more spiral coil(s) 102 can be disposed within the chamber 116. In some embodiments, one or more spiral coil(s) 102 disposed in the cavity 118 can be configured to receive RF energy signals emitted by the sample 101, and one or more spiral coil(s) 102 disposed within the chamber 116 can be configured to transmit RF energy to the sample 101. In some embodiments, one or more spiral coil(s) 102 disposed within the cavity 118 and/or one or more spiral coil(s) disposed within the chamber 116 can be configured to transmit and receive RF energy to and from the sample 101.

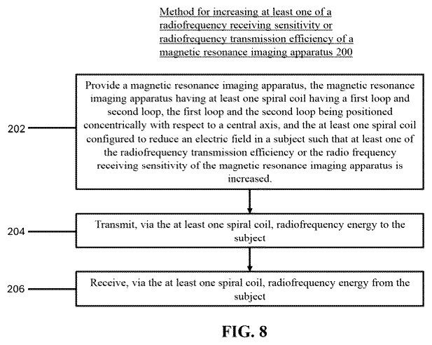

Referring to FIG. 8, an exemplary method 200 for increasing at least one of a radiofrequency (RF) receiving sensitivity or RF transmission efficiency of a magnetic resonance imaging (MRI) apparatus is provided. The method 200 can utilize any of the above-described embodiments and/or elements of the MRI apparatus 100 for increasing at least one of RF receiving sensitivity or RF transmission efficiency, the details of which are not repeated here in the interest of brevity. The method 200 can include a first step 202, providing a MRI apparatus. The MRI apparatus can include at least one spiral coil having a first loop and a second loop positioned concentrically with respect to a central axis. The at least one spiral coil can be configured to reduce an electric field in a sample such that at least one of a radiofrequency transmission efficiency or a radiofrequency receiving sensitivity of the magnetic resonance imaging apparatus is increased. The method 200 can include a second step 204, transmitting, via the at least one spiral coil, RF energy to the sample. The method 200 can include a third step 206, receiving, via the at least one spiral coil, RF energy from the sample. In some embodiments, the method 200 can include one of steps 204 or 206. In some embodiments, the method 200 can include both of steps 204 and 206.

The following disclosure discusses exemplary implementations, method, and test data related to the same.

### EXAMPLES

### Synopsis

We investigated the denoising effect of employing double-loop spiral coil (DLSC) versus the conventional single loop surface coil in the presence of ultrahigh dielectric constant materials (uHDCM). The DLSC confines the conservative E-field distribution between the loops which as a result reduces the E-field in the sample, therefore reduces the noise level. Reduction of conservative E-Field makes the non-conservative E-field more dominant in the sample, which synergistically with the noise reduction allows increasing transmit efficiency and receive sensitivity for MRI application.

### Summary of Main Findings

In comparison with a traditional single-loop surface coil, the double-loop spiral coil (DLSC) has advantages to reduce the E-field in the imaging sample and improve RF coil transmission and reception efficiencies for MRI application.

### INTRODUCTION

A large denoising effect with the ultrahigh dielectric constant materials (uHDCM) and a conventional single-loop surface coil was demonstrated for Na imaging at 10.5T and O imaging at 7T magnets. It was shown previously that B₁ field can be enhanced several folds in the sample by induced strong displacement current in uHDCM next to the sample. The displacement current in uHDCM is produced by non-conservative E field induced by the coil. The E field by the RF coil in the sample consists of both conservative and non-conservative components, both of which produce losses and generate noises in a conductive sample. This decomposition of the E-field is shown in Eq. 1:

$$E = {- ∇ φ}^{\overbrace{\to }^{conservative}} - \overbrace{j ω \vec{A}}^{non - conservative}$$

where φ is the electric scalar potential, \vec{A} is the vector potential, and ω is the angular frequency. It was shown that employing uHDCM not only increase the induced displacement current and therefore enhance the B₁ field but also reduces the conservative field and decreases the MR imaging noise. For further reduction of conservative E-field, in this work we propose using dual loop spiral surface coil and therefore achieving more enhancement of RF magnetic transmission field (B₁⁺) efficiency and reception field (B₁⁻, Rx-sensitivity).

### Experimental Methods

### Simulation Setup

We performed the numerical calculation of Maxwell equations by using CST Microwave Studio as a full-wave simulation software. The setup (FIGS. 9A-9C) includes a 15 cm diameter coil shaped from a copper wire, a cylindrical uHDCM disk (FIGS. 9B-C), and a cylindrical water phantom placed on top of the uHDCM disk. The thickness and diameter of the uHDCM disk is chosen as 2.1 cm and 8 cm, respectively. The relative permittivity and conductivity of the water phantom with the diameter of 9 cm and height of 7 cm, is chosen εᵣ=80 and σ=1.5 S/m, respectively. FIG. 9A shows a single loop coil and a water phantom, FIG. 9B shows a single loop coil, a uHDCM disk, and a water phantom, and FIG. 9C shows a double-loop spiral coil, a uHDCM disk, and a water phantom. The single loop coil and DLSC radii are chosen 7.5 cm and, matched to 50 Ohm and tuned at 63 MHz (close to ¹⁷O Larmor frequency at 10.5T). To perform our investigation, we studied the field at employing uHDCM with εᵣ=6000 and then compared the value of normal electric field at the phantom and the Rx-sensitivity of the coil for three cases of investigation.

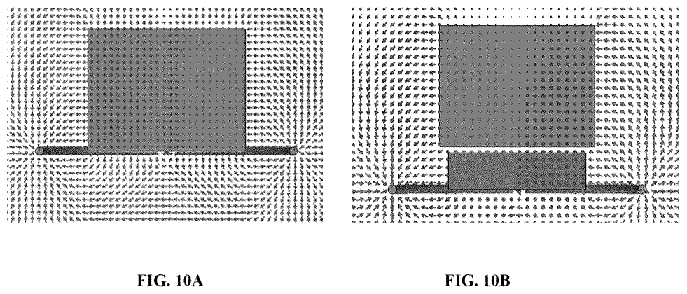

FIGS. 10A-B and 11A-B show electric field maps extracted in vector plot form on the coronal plane for a single loop coil (FIG. 10A), a single loop coil with a uHDCM disk resonator (FIG. 10B), a double loop spiral coil (FIG. 11A) and a double loop spiral coil with a uHDCM disk resonator (FIG. 11B), each with a phantom placed adjacent the coil and/or uHDCM disk resonator.

### Rx Sensitivity Gain and Noise Level (NL) Reduction

The Rx-sensitivity is calculated by dividing the B₁⁻ over the square root of the dissipated power within the phantom at 63 MHz. The normalized noise level (NNL) can be calculated using Eq. 2:

$$NNL = \sqrt{\frac{\int σ {\vert E \vert}^{2} d v}{a v g ( {B}_{1}^{{+}^{2}} )}} , \qquad ( 2 )$$

where σ is the conductivity of the phantom, and E is the electric field. The integration is performed in the whole volume of the phantom. Based on (Eq. 2), by the reduction of E-field in the phantom, the NNL value received at the loop will reduce and therefore the Rx sensitivity increases.

### Results and Discussion

As shown in FIG. 10A, the E-field in the sample region for the baseline consists of a set of parallel lines indicating a conservative field. In the presence of the phantom the E-field vectors in the phantom follow circular lines, indicating the field is dominantly non-conservative inside the phantom, while outside the phantom, the E-field is dominantly conservative. The conservative E-field is produced by the surface charges of the coil and those of the phantom with opposite charges. The E-field intensity is also decreased within the phantom.

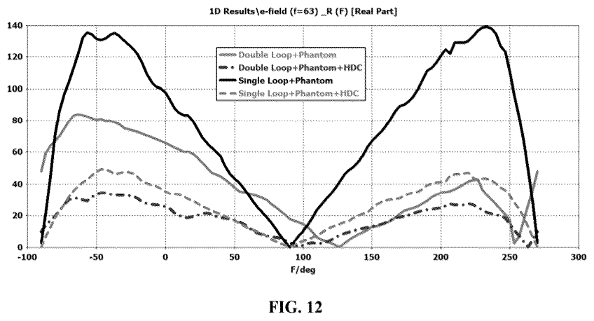

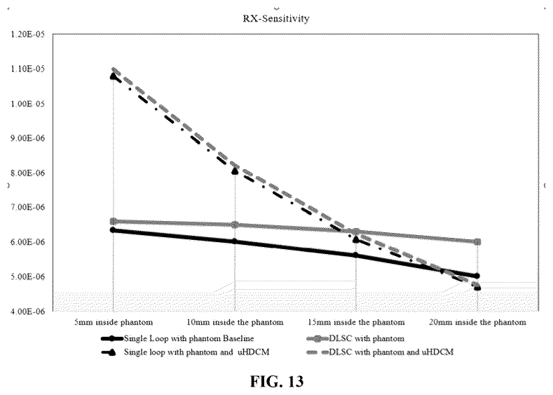

However, in the presence of uHDCM (FIG. 10B), we observed that the conservative E-field at the boundary of the phantom is reduced compared to FIG. 10A. Similarly, the conservative E-field at the boundary of the phantom is reduced in the presence of the double loop spiral coil (FIGS. 11A-B) as compared to the single loop coil (FIGS. 10A-B), and further reduced in the presence of the double loop spiral coil and the uHDCM (FIG. 11B) as compared to the double loop spiral coil (FIG. 11A). The line plot in FIG. 12 quantitatively reveals this reduction of conservative field. We found the least E-field at employing the DLSC, which leads to a significant reduction of the noise level. Table 1 (below) and FIG. 13 show the Rx-sensitivity for all three cases in small planes parallel to the coil position inside the phantom. The Rx-sensitivity is enhanced by using the uHDCM disk and double-loops spiral coil in from the surface to 15 mm depth inside the phantom. Our future investigation would be the array of new design coils to further enhance sensitivity for the whole phantom volume.

TABLE 1
RX-Sensitivity

| | 5 mm inside | 10 mm inside | 15 mm inside | 20 mm inside |
|---|---|---|---|---|
| | phantom | the phantom | the phantom | the phantom |
| Single Loop with | 6.34e−06 | 6e−06 | 5.6e−06 | 5e−06 |
| phantom Baseline | | | | |
| DLSC with | 6.6e−6 | 6.5e−6 | 6.3e−6 | 6.2e−6 |
| phantom | | | | |
| Single loop with | 1.08e−05 | 8.05e−06 | 6.07e−06 | 4.7e−06 |
| phantom and | | | | |
| uHDCM | | | | |
| DLSC with | 1.1e−05 | 8.21e−06 | 6.23e−06 | 4.75e−06 |
| phantom and | | | | |
| uHDCM | | | | |

### CONCLUSION

With computer modeling under an identical setup, we demonstrated that although the uHDCM reduces the conservative E-field and noise level, employing the DLSC can be useful to achieve even more reduction of conservative E-field and therefore enhance the B₁⁺ efficiency in the phantom and Rx sensitivity at the coil loop. This property leads to a prominent de-noising effect and can benefit the many MRI applications.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purpose of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the compositions, materials, apparatuses, and methods of using and making the same disclosed herein have been discussed and illustrated, it is to be distinctly understood that the invention is not limited thereto but may otherwise be variously embodied and practiced within the scope of the following claims.

What is claimed is:

1. A magnetic resonance imaging apparatus comprising:
at least one spiral coil comprising a first loop and a second loop, wherein the first loop and the second loop are positioned concentrically with respect to a central axis and spaced apart to define a gap intermediate the first loop and the second loop;
wherein the at least one spiral coil is configured to reduce its conservative electric field induced in a sample to be imaged such that at least one of a radiofrequency transmission efficiency or a radiofrequency receiving sensitivity of the apparatus is increased.

2. The magnetic resonance imaging apparatus of claim 1, wherein reducing the conservative electric field induced in the sample comprises confining the conservative electric field between the first loop and the second loop such that electric field generated within the sample is dominantly non-conservative.

3. The magnetic resonance imaging apparatus of claim 2, wherein the conservative electric field is confined within the gap defined intermediate the first loop and the second loop.

4. The magnetic imaging apparatus of claim 1, wherein the at least one spiral coil is further configured to reduce a noise level in the subject.

5. The magnetic resonance imaging apparatus of claim 1, further comprising a plurality of spiral coils.

6. The magnetic resonance imaging apparatus of claim 1, further comprising at least one high dielectric constant material disk disposed within the at least one spiral coil, wherein the at least one high dielectric constant material disk is configured to increase at least one of the radiofrequency transmission efficiency or the radiofrequency receiving sensitivity of the magnetic resonance imaging apparatus.

7. The magnetic resonance imaging apparatus of claim 6, wherein the high dielectric constant material disk comprises a disk resonator with a disk resonance frequency equal to a coil resonance frequency of the at least one spiral coil.

8. The magnetic resonance imaging apparatus of claim 6, wherein the high dielectric constant material disk comprises at least one ultrahigh dielectric material.

9. A method for increasing at least one of a radiofrequency receiving sensitivity or radiofrequency transmission efficiency of a magnetic resonance imaging apparatus, the method comprising:
providing the magnetic resonance imaging apparatus comprising:
at least one spiral coil comprising a first loop and a second loop, wherein the first loop and the second loop are positioned concentrically with respect to a central axis and spaced apart to define a gap intermediate the first loop and the second loop, wherein the at least one spiral coil is configured to reduce its conservative electric field induced in a sample such that at least one of the radiofrequency transmission efficiency or the radiofrequency receiving sensitivity of the magnetic resonance imaging apparatus is increased;
transmitting, via the spiral coil, radiofrequency energy to the sample; and/or
receiving, via the spiral coil, radiofrequency energy from the sample.

10. The method of claim 9, wherein reducing the conservative electric field induced in the sample comprises confining the conservative electric field induced by the at least one spiral coil between the first loop and the second loop such that a dominantly non-conservative electric field is generated within the sample.

11. The method of claim 10, wherein the conservative electric field is confined within the gap defined intermediate the first loop and the second loop.

12. The method of claim 9, wherein the magnetic resonance imaging apparatus further comprises a plurality of spiral coils.

13. The method of claim 9, wherein the magnetic imaging apparatus further comprises at least one high dielectric constant material disk disposed within the at least one spiral coil, wherein the at least one high dielectric constant material disk is configured to increase at least one of the radiofrequency transmission efficiency or the radiofrequency receiving sensitivity of the magnetic resonance imaging apparatus.

14. The method of claim 13, wherein the high dielectric constant material disk comprises a disk resonator with a disk resonance frequency equal to a coil resonance frequency of the at least one spiral coil.
